(19) United States
(12) Patent Application Publication
(10) Pub. No.: US 2026/0124414 A1
(43) Pub. Date: May 7, 2026

(54) INTEGRATED BREATHING CIRCUIT, CONTROL METHOD THEREOF AND ELECTRONIC SYSTEM

(71) Applicant: Telesair, Inc., Irvine, CA (US)

(72) Inventors: Chi Wai CHOY, Irvine, CA (US); Nguyen NGUYEN, Yorba Linda, CA (US); Sreevalsan Sanathanan MENON, Santa Ana, CA (US)

(73) Assignee: Telesair, Inc., Irvine, CA (US)

(21) Appl. No.: 18/934,536

(22) Filed: Nov. 1, 2024

(51) Int. Cl. A61M 16/00; A61M 16/10

(52) U.S. Cl. CPC A61M 16/022; A61M 16/0003; A61M 16/109; A61M 16/1095; A61M 2202/0007; A61M 2202/0208; A61M 2205/3327; A61M 2205/3368; A61M 2205/3653; A61M 2205/50

(57) ABSTRACT

An integrated breathing circuit, a control method thereof and an electronic system. The integrated breathing circuit includes: a first circuit, including two heater wires coupled together; and a second circuit, including a temperature sensor and two wires connected with the temperature sensor; where the heater wires are configured to regulate and maintain a temperature within the integrated breathing circuit, and the temperature sensor is configured to measure the temperature within the integrated breathing circuit.

### TECHNICAL FIELD

The present disclosure relates to the technical field of medical devices and, in particular, to an integrated breathing circuit, a control method thereof and an electronic system.

### BACKGROUND

For patients who require the use of respiratory aids, providing an airflow with an appropriate temperature is very beneficial. However, interference between a temperature sensor and a heater affects the accuracy of temperature measurement and further affects the control of the airflow temperature.

This background information is provided to reveal information believed by the applicant to be of possible relevance to the present disclosure. No admission is necessarily intended, nor should be construed, that any of the preceding information constitutes prior art against the present disclosure.

### SUMMARY

The present disclosure provides an integrated breathing circuit, a control method thereof and an electronic system. According to the breathing circuit, the control method thereof and the electronic system, the heater and temperature sensor are decoupled by setting them in different circuits, the interference of the heater on the temperature sensor can be reduced, thereby improving accurate temperature measurements in varying airflow scenarios.

In a first aspect, an embodiment of the present disclosure provides an integrated breathing circuit, including:
a first circuit, including two heater wires coupled together; and
a second circuit, including a temperature sensor and two wires connected with the temperature sensor;
where the heater wires are configured to regulate and maintain a temperature within the integrated breathing circuit, and the temperature sensor is configured to measure the temperature within the integrated breathing circuit.

In a possible implementation, the integrated breathing circuit further includes: a shield part, configured to isolate the first circuit and the second circuit.

In a possible implementation, the shield part is an insulated part.

In a possible implementation, a size of the shield part and/or a placement manner of the shield part is based on a distance between the first circuit and the second circuit.

In a possible implementation, an end of the integrated breathing circuit is connected with a control device, and the control device is configured to receive temperature data from the temperature sensor and control the heater wires and the temperature sensor to operate at different times based on the temperature data.

In a possible implementation, the control device is configured to determine when to activate or deactivate the heater wires based on the temperature data.

In a possible implementation, the control device is configured to determine when to activate or deactivate the heater wires based on the temperature data and a target temperature parameter.

In a possible implementation, the target temperature parameter includes a desired temperature range.

In a possible implementation, when a first temperature value indicated by the temperature data is greater than an upper limit value of the desired temperature range, the control device is configured to deactivate the heater wires until a second temperature value indicated by the temperature data is less than a lower limit value of the desired temperature range, and control the temperature sensor to perform temperature sampling during the heater wires being deactivated; and/or
when a third temperature value indicated by the temperature data is less than a lower limit value of the desired temperature range, the control device is configured to activate and deactivate the heater wires at a frequency, and control the temperature sensor to perform temperature sampling during the heater wires being deactivated.

In a possible implementation, the control device is configured to activate or deactivate the heater wires by regulating application of power into the first circuit.

In a possible implementation, the desired temperature range is a preset range, or is configured by a user.

In a possible implementation, the control device is a breathing device, and the breathing device is used for humidification and/or oxygen supply.

In a possible implementation, both of the first circuit and the second circuit is arranged in a conduit.

In a second aspect, an embodiment of the present disclosure provides a control method, applied to a control device to control an integrated breathing circuit, where the integrated breathing circuit includes: a first circuit, including two heater wires coupled together; and a second circuit, including a temperature sensor and two wires connected with the temperature sensor; where the heater wires are configured to regulate and maintain a temperature within the integrated breathing circuit, and the temperature sensor is configured to measure the temperature within the integrated breathing circuit; and the method includes:
receiving temperature data from the temperature sensor in the integrated breathing circuit; and
controlling, based on the temperature data, the heater wires and the temperature sensor in the integrated breathing circuit to operate at different times.

In a possible implementation, the method further includes:
determining when to activate or deactivate the heater wires based on the temperature data.

In a possible implementation, the determining when to activate or deactivate the heater wires based on the temperature data includes:
determining when to activate or deactivate the heater wires based on the temperature data and a target temperature parameter.

In a possible implementation, the target temperature parameter includes a desired temperature range.

In a possible implementation, the determining when to activate or deactivate the heater wires based on the temperature data and the target temperature parameter includes: determining whether a first temperature value indicated by the temperature data is greater than an upper limit value of the desired temperature range; and the controlling, based on the temperature data, the heater wires and the temperature sensor in the integrated breathing circuit to operate at different times includes: when the first temperature value is greater than the upper limit value, deactivating the heater wires until a second temperature value indicated by the temperature data is less than a lower limit value of the desired temperature range; and controlling the temperature sensor to perform temperature sampling during the heater wires being deactivated, and/or
the determining when to activate or deactivate the heater wires based on the temperature data and the target temperature parameter includes: determining whether a third temperature value indicated by the temperature data is less than a lower limit value of the desired temperature range; and the controlling, based on the temperature data, the heater wires and the temperature sensor in the integrated breathing circuit to operate at different times includes: activating and deactivating the heater wires at a frequency when the third temperature value is less than the lower limit value; and controlling the temperature sensor to perform temperature sampling during the heater wires being deactivated.

In a possible implementation, the activating or deactivating the heater wires includes regulating application of power into the first circuit.

In a third aspect, an embodiment of the present disclosure provides an electronic system, including an integrated breathing circuit and a control device; where
the integrated breathing circuit includes: a first circuit, including two heater wires coupled together; and a second circuit, including a temperature sensor and two wires connected with the temperature sensor; where the heater wires are configured to regulate and maintain a temperature within the integrated breathing circuit, and the temperature sensor is configured to measure the temperature within the integrated breathing circuit.

It should be understood that the content described in this section is not intended to identify the key or important features of the embodiments of the present disclosure, nor to limit the scope of the present disclosure. Other features of the present disclosure will be easily understood through the following description.

### BRIEF DESCRIPTION OF THE DRAWINGS

The drawings are used for a better understanding of the present solution but do not constitute any limitation on the present disclosure. Throughout the drawings, identical or similar reference numbers designate identical or similar elements.

FIG. 1 is an exemplary schematic diagram of a respiratory support system according to an embodiment of the present disclosure.

FIG. 2 is a schematic structural diagram of an integrated breathing circuit according to an embodiment of the present disclosure.

FIG. 3 is a schematic diagram of a control method according to an embodiment of the present disclosure.

FIG. 4 is a schematic structural diagram of a control apparatus according to an embodiment of the present disclosure.

FIG. 5 is a schematic structural diagram of an electronic device according to an embodiment of the present disclosure.

### DETAILED DESCRIPTION OF THE EMBODIMENTS

In the following description, reference is made to the accompanying figures, which form part of the disclosure, and which show, by way of illustration, specific aspects of embodiments of the present disclosure or specific aspects in which embodiments of the present disclosure may be used. It is understood that embodiments of the present disclosure may be used in other aspects and include structural or logical changes not depicted in the figures. The following detailed description, therefore, is not to be taken in a limiting sense, and the scope of the present disclosure is defined by the appended claims.

The term “include” used herein and its variations are open inclusion, that is, “include but not limited to”. The term “based on” means “at least partly based on”. The term “an embodiment” represents “at least one embodiment”; the term “another embodiment” represents “at least one another embodiment”; and the term “some embodiments” represents “at least some embodiments”. Related definitions of other terms will be provided in the following. It should be noted that concepts such as “first”, and “second” mentioned in the present disclosure are merely used to distinguish different apparatuses, modules, or units, but not to limit the sequence or interdependency of functions executed by these apparatuses, modules, or units.

It should be noted that the singular or plural modification mentioned in the present disclosure is illustrative and not restrictive, and those skilled in the art should understand that it should be understood as “one or more” unless clearly defined in the context otherwise.

For patients who require the use of respiratory aids, providing an airflow with an appropriate temperature is very beneficial. However, interference between a temperature sensor and a heater affects the accuracy of temperature measurement and further affects the control of the airflow temperature.

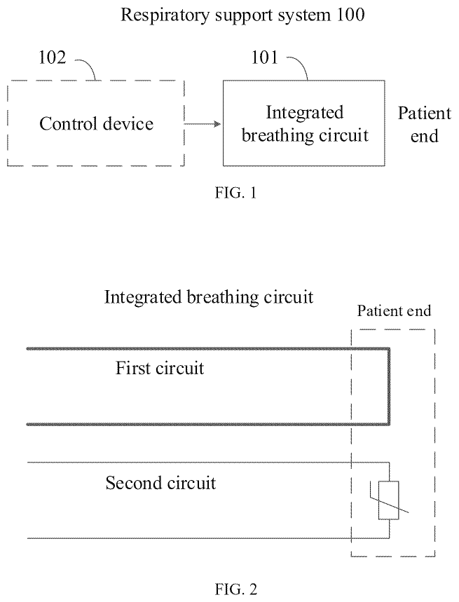

FIG. 1 is an exemplary schematic diagram of a respiratory support system according to an embodiment of the present disclosure. As shown in FIG. 1, the respiratory support system includes an integrated breathing circuit, which control and/or regulates a temperature of an airflow transferred to a patient. The airflow, whose temperature is regulated, is provided to the patient via a patient end.

In some cases, the respiratory support system further includes a control device, which may be a breathing device. In an implementation, the breathing device may be used to provide airflow for assisting breathing, where the airflow may be with a high flow rate or a low flow rate, which is not limited in the present application. For example, the breathing device may be used for humidification, for oxygen supply, and etc. More specifically, the breathing device may be a Bonhawa Humidifier.

FIG. 2 is a schematic structural diagram of an integrated breathing circuit according to an embodiment of the present disclosure. As shown in FIG. 2, the integrated breathing circuit includes a first circuit and a second circuit, where the first circuit includes two heater wires coupled together; and the second circuit includes a temperature sensor and two wires connected with the temperature sensor.

Among them, the heater wires are configured to regulate and maintain a temperature within the integrated breathing circuit, and the temperature sensor is configured to measure the temperature within the integrated breathing circuit.

In one implementation, the two heating wires may be circuits containing resistance wires, which achieve heating function after applying power.

It is obvious that the integrated breathing circuit is a passive configuration, specifically, the breathing circuit employs a four-wire passive configuration, whereas two wires are coupled together to provide heater functionality and two wires are interfacing to a temperature sensor.

By decoupling the heater and temperature sensor lines, by setting them in different circuits, the interference of the heater wires on the temperature sensor (including but not limited to electrical noise, heat transfer, and etc.) can be reduced, thereby improving accurate temperature measurements in varying airflow scenarios. In addition, the four wire structure is easy to achieve separate control of the heater wires and temperature sensor.

The temperature sensor includes but is not limited to a thermistor, a thermocouple, a resistance temperature detector (RTD), an infrared sensor, or an integrated breathing circuit sensor.

In an implementation, the integrated breathing circuit further includes a shield part, which is configured to isolate the first circuit and the second circuit.

By setting the shield part to isolate the first circuit and the second circuit, electrical noises during temperature sampling while the heater wires are working can be mitigated, thereby reducing interference between the temperature sensor and the heater wires and resulting in more accurate temperature data. Furthermore, the shield part between the first circuit and the second circuit creates a certain degree of physical separation, thereby reducing the impact of heat transfer from the heater wires to the temperature sensor.

In some cases, the shield part may be an insulated part. By using the insulated part to isolate the first circuit and the second circuit, electrical noises during an active heating phase (i.e., during the heater wires working) can be minimized, thereby reducing the interference between the temperature sensor and the heater.

In some cases, a size of the shield part and/or a placement manner of the shield part is based on a distance between the first circuit and the second circuit.

In an implementation, an end of the integrated breathing circuit is connected with a control device, and the control device is configured to receive temperature data from the temperature sensor and control the heater wires and the temperature sensor to operate at different times based on the temperature data.

Specifically, the control device controls the heater wires by regulating an application of a power into the first circuit. And the control device controls and interfaces to the temperature sensor by applying a power into the second circuit.

The heater and the temperature sensor are controlled to operate at different times based on the temperature data received from the temperature sensor, that is, a temperature sampling of the temperature sensor is executed when the heater wires are disabled, in this way, each time the temperature sensor works, electrical noise generated when power is applied to the first circuit including the heater wires can be completely eliminated.

The control device is further configured to determine when to activate or deactivate the heater wires based on the temperature data.

Furthermore, the control device is configured to determine when to activate or deactivate the heater wires based on the temperature data and a target temperature parameter. Among them, the target temperature parameter includes a desired temperature range.

Exemplarily, the desired temperature range may be a temperature range that is comfortable for the human body. For example, the desired temperature range may be 30 to 37 degrees Celsius, or 31 to 35 degrees Celsius. The desired temperature range may be a preset range, or may be configured by a user.

When a first temperature value indicated by the temperature data is greater than an upper limit value of the desired temperature range, the control device deactivates the heater wires until a second temperature value indicated by the temperature data is less than a lower limit value of the desired temperature range, and control the temperature sensor to perform temperature sampling during the heater wires being deactivated.

Alternatively, when a third temperature value indicated by the temperature data is less than a lower limit value of the desired temperature range, the control device is configured to activate and deactivate the heater wires at a frequency, and control the temperature sensor to perform temperature sampling during the heater wires being deactivated.

Exemplarily, taken the desired temperature range of 30 to 37 degrees Celsius as an example, when the first temperature value indicated by the temperature data is 37.2 degrees Celsius, which is greater than the upper limit value (that is, 37 degrees Celsius in this example) of the desired temperature range, the control device deactivates the heater wires and waits for subsequent temperature values indicated by the temperature data measured by the temperature sensor. Once a temperature value indicated by the temperature data falls to 29.9 degrees Celsius, which is less than the lower limit value (that is, 30 degrees Celsius in this example) of the desired temperature range, the control device activates and deactivates the heater wires at a frequency. And the temperature sensor is controlled to perform temperature sampling during the periods when the heater wires are deactivated.

As stated above, the control device controls the heater wires by regulating an application of a power into the first circuit, thus the control device is configured to activate or deactivate the heater wires by regulating application of power to the first circuit.

In an implementation, the control device is a breathing device, and the breathing device is used for humidification and/or oxygen supply.

In an implementation, both the first circuit and the second circuit are arranged in a conduit. By integrating the circuit including two heater wires coupled together and the circuit including the temperature sensor into one conduit, an interface layout on the control device can be simplified, which makes the respiratory support system more convenient for users to use. Furthermore, integrating of the circuit including the heater wires and the circuit including the temperature sensor ensures to avoid mixing of temperature sensors and heaters from different manufacturers in actual use, thereby fully ensuring compatibility between the heating function and the temperature measuring function, and ensuring the safety of the patient.

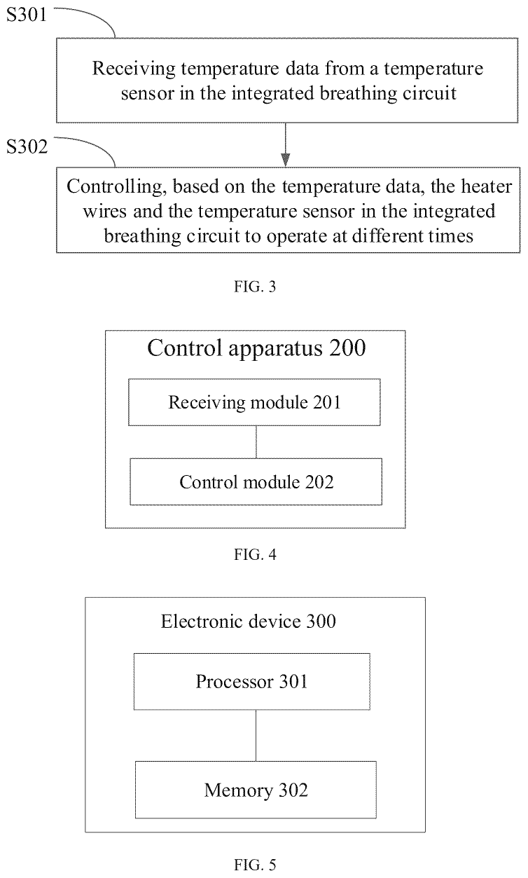

FIG. 3 is a schematic diagram of a control method according to an embodiment of the present disclosure. Since the principles of the control method and the integrated breathing circuit to solve the problem are similar, the definition and the explanation of the same terminologies used in the description of the control method can be referred to that in the description of the integrated breathing circuit, and the repetitive details will not be repeated. The control method shown in FIG. 3 is applied to a control device to control an integrated breathing circuit, includes:
S301: receiving temperature data from the temperature sensor in the integrated breathing circuit; and
S302: controlling, based on the temperature data, the heater wires and the temperature sensor in the integrated breathing circuit to operate at different times.

Among them, the integrated breathing circuit includes: a first circuit, including two heater wires coupled together; and a second circuit, including a temperature sensor and two wires connected with the temperature sensor; where the heater wires are configured to regulate and maintain a temperature within the integrated breathing circuit, and the temperature sensor is configured to measure the temperature within the integrated breathing circuit.

In an implementation, the method further includes:
determining when to activate or deactivate the heater wires based on the temperature data.

In an implementation, the determining when to activate or deactivate the heater wires based on the temperature data includes:
determining when to activate or deactivate the heater wires based on the temperature data and a target temperature parameter.

In an implementation, the target temperature parameter includes a desired temperature range.

In an implementation, the determining when to activate or deactivate the heater wires based on the temperature data and the target temperature parameter includes: determining whether a first temperature value indicated by the temperature data is greater than an upper limit value of the desired temperature range; and S302 includes: when the first temperature value is greater than the upper limit value, deactivating the heater wires until a second temperature value indicated by the temperature data is less than a lower limit value of the desired temperature range; and controlling the temperature sensor to perform temperature sampling during the heater wires being deactivated; and/or
the determining when to activate or deactivate the heater wires based on the temperature data and the target temperature parameter includes: determining whether a third temperature value indicated by the temperature data is less than a lower limit value of the desired temperature range; and S302 includes: activating and deactivating the heater wires at a frequency when the third temperature value is less than the lower limit value; and controlling the temperature sensor to perform temperature sampling during the heater wires being deactivated.

In an implementation, the activating or deactivating the heater wires includes regulating application of power into the first circuit.

The control method provided by the present application proposes a novel solution for improved accuracy and performance of the temperature control of a breathing circuitry. By focusing on selective temperature sampling and placing a crucial emphasis on minimizing heat transfer effects, especially in dynamic airflow conditions, this innovation offers improved accuracy and performance, marking a significant advancement in breathing circuit technology.

The present disclosure further provides a control apparatus that can be configured to perform the control method for controlling an integrated breathing circuit mentioned above. Since the principles of the method and the apparatus to solve the problem are similar, the definition and the explanation of the same terminologies used in the description of the control apparatus can be referred to that in the description of the control method, and the repetitive details will not be repeated. FIG. 4 is a schematic structural diagram of a control apparatus according to an embodiment of the present disclosure. As shown in FIG. 4, the control apparatus 200 can include:
an receiving module 201, configured to receive temperature data from the temperature sensor in the integrated breathing circuit; and
a control module 202, configured to control the heater and the temperature sensor in the integrated breathing circuit to operate at different times based on the temperature data.

Among them, the integrated breathing circuit includes: a first circuit, including two heater wires coupled together; and a second circuit, including a temperature sensor and two wires connected with the temperature sensor; where the heater wires are configured to regulate and maintain a temperature within the integrated breathing circuit, and the temperature sensor is configured to measure the temperature within the integrated breathing circuit.

In an implementation, the control apparatus 200 further includes a determining module, configured to determine when to activate or deactivate the heater wires based on the temperature data.

In an implementation, the determining module is configured to determine when to activate or deactivate the heater wires based on the temperature data and a target temperature parameter.

In an implementation, the target temperature parameter includes a desired temperature range.

In an implementation, the determining module is specifically configured to: determine whether a first temperature value indicated by the temperature data is greater than an upper limit value of the desired temperature range; and the control module 202 is specifically configured to when the first temperature value is greater than the upper limit value, deactivate the heater wires until a second temperature value indicated by the temperature data is less than a lower limit value of the desired temperature range and control the temperature sensor to perform temperature sampling during the heater wires being deactivated; and/or
the determining module is specifically configured to: determine whether a third temperature value indicated by the temperature data is less than a lower limit value of the desired temperature range; and the control module 202 is specifically configured to activate and deactivate the heater wires at a frequency when the third temperature value is less than the lower limit value; and control the temperature sensor to perform temperature sampling during the heater wires being deactivated.

In an implementation, the control module 202 is specifically configured to regulating application of power into the first circuit.

The present disclosure further provides an electronic system (i.e., the respiratory support system 100 as shown in FIG. 1), which includes an integrated breathing circuit and a control device; where the integrated breathing circuit includes: a first circuit, two heater wires coupled together; and a second circuit, including a temperature sensor and two wires connected with the temperature sensor; where the heater wires are configured to regulate and maintain a temperature within the integrated breathing circuit, and the temperature sensor is configured to measure the temperature within the integrated breathing circuit.

The present disclosure further provides a non-transitory computer-readable storage medium, which stores therein computer-executable instructions which, when being executed by one or more processors, implement the control method according to embodiments of the present disclosure.

The present disclosure further provides a computer program, when the computer program is executed by one or more processors, implements the control method according to embodiments of the present disclosure.

The present disclosure further provides a computer program product, which stores thereon computer executable instructions which, when being executed by one or more processors, implements the control method according to embodiments of the present disclosure.

In one or more examples, the functions described may be implemented in hardware, software, firmware, or any combination thereof. For example, the functions may be implemented by one or more processors, such as one or more application-specific integrated circuits (ASICs), field programmable logic arrays (FPGAs), or other equivalent integrated or discrete logic circuitry. Accordingly, the term “processor,” as used herein may refer to any of the foregoing structures or any other structure suitable for the implementation of the techniques described herein. In addition, the techniques could be fully implemented in one or more circuits or logic elements.

Of course, the devices and components shown in the drawings may include further elements that are not shown in the drawings. The functions of the foreign object monitoring apparatus described in the specification can be realized by a circuit, which includes a subcircuit or a combination of a plurality of subcircuits, that is, the modules (the obtaining module and the determining module) described in the specification can be implemented as a subcircuit or a combination of a plurality of subcircuits.

The foregoing detailed description has been presented for purposes of illustration and description. It is not intended to be exhaustive or to limit the subject matter claimed herein to the precise form(s) disclosed. Many modifications and variations are possible in light of the above teachings. The described embodiments were chosen in order to best explain the principles of the disclosed technology and its practical application to thereby enable others skilled in the art to best utilize the technology in various embodiments and with various modifications as are suited to the particular use contemplated. Those embodiments with various modifications are within the range and scope of the following claims.

What is claimed is:

1. An integrated breathing circuit, comprising:
a first circuit comprising two heater wires coupled together; and
a second circuit comprising a temperature sensor and two wires connected with the temperature sensor;
wherein the heater wires are configured to regulate and maintain a temperature within the integrated breathing circuit, and the temperature sensor is configured to measure the temperature within the integrated breathing circuit.

2. The integrated breathing circuit according to claim 1, further comprising:
a shield part, configured to isolate the first circuit and the second circuit.

3. The integrated breathing circuit according to claim 2, wherein the shield part is an insulated part.

4. The integrated breathing circuit according to claim 2, wherein a size of the shield part and/or a placement manner of the shield part is based on a distance between the first circuit and the second circuit.

5. The integrated breathing circuit according to claim 1, wherein an end of the integrated breathing circuit is connected with a control device, and the control device is configured to receive temperature data from the temperature sensor and control the heater wires and the temperature sensor to operate at different times based on the temperature data.

6. The integrated breathing circuit according to claim 5, wherein the control device is configured to determine when to activate or deactivate the heater wires based on the temperature data.

7. The integrated breathing circuit according to claim 6, wherein the control device is configured to determine when to activate or deactivate the heater wires based on the temperature data and a target temperature parameter.

8. The integrated breathing circuit according to claim 7, wherein the target temperature parameter comprises a desired temperature range.

9. The integrated breathing circuit according to claim 8, wherein
when a first temperature value indicated by the temperature data is greater than an upper limit value of the desired temperature range, the control device is configured to deactivate the heater wires until a second temperature value indicated by the temperature data is less than a lower limit value of the desired temperature range, and control the temperature sensor to perform temperature sampling during the heater wires being deactivated; and/or
when a third temperature value indicated by the temperature data is less than a lower limit value of the desired temperature range, the control device is configured to activate and deactivate the heater wires at a frequency, and control the temperature sensor to perform temperature sampling during the heater wires being deactivated.

10. The integrated breathing circuit according to claim 9, wherein the control device is configured to activate or deactivate the heater wires by regulating application of power into the first circuit.

11. The integrated breathing circuit according to claim 8, wherein the desired temperature range is a preset range, or is configured by a user.

12. The integrated breathing circuit according to claim 5, wherein the control device is a breathing device, and the breathing device is used for humidification and/or oxygen supply.

13. The integrated breathing circuit according to claim 1, wherein both of the first circuit and the second circuit is arranged in a conduit.

14. A control method, applied to a control device to control an integrated breathing circuit, wherein
the integrated breathing circuit comprises: a first circuit comprising two heater wires coupled together; and a second circuit comprising a temperature sensor and two wires connected with the temperature sensor; wherein the heater wires are configured to regulate and maintain a temperature within the integrated breathing circuit, and the temperature sensor is configured to measure the temperature within the integrated breathing circuit;
the method comprising:
receiving temperature data from the temperature sensor in the integrated breathing circuit; and
controlling, based on the temperature data, the heater wires and the temperature sensor in the integrated breathing circuit to operate at different times.

15. The method according to claim 14, further comprising:
determining when to activate or deactivate the heater wires based on the temperature data.

16. The method according to claim 15, wherein the determining when to activate or deactivate the heater wires based on the temperature data comprises:
determining when to activate or deactivate the heater wires based on the temperature data and a target temperature parameter.

17. The method according to claim 16, wherein the target temperature parameter comprises a desired temperature range.

18. The method according to claim 17, wherein
the determining when to activate or deactivate the heater wires based on the temperature data and the target temperature parameter comprises: determining whether a first temperature value indicated by the temperature data is greater than an upper limit value of the desired temperature range; and the controlling, based on the temperature data, the heater wires and the temperature sensor in the integrated breathing circuit to operate at different times comprises: when the first temperature value is greater than the upper limit value, deactivating the heater wires until a second temperature value indicated by the temperature data is less than a lower limit value of the desired temperature range; and controlling the temperature sensor to perform temperature sampling during the heater wires being deactivated, and/or
the determining when to activate or deactivate the heater wires based on the temperature data and the target temperature parameter comprises: determining whether a third temperature value indicated by the temperature data is less than a lower limit value of the desired temperature range; and the controlling, based on the temperature data, the heater wires and the temperature sensor in the integrated breathing circuit to operate at different times comprises: activating and deactivating the heater wires at a frequency when the third temperature value is less than the lower limit value; and controlling the temperature sensor to perform temperature sampling during the heater wires being deactivated.

19. The method according to claim 18, wherein the activating or deactivating the heater wires comprises regulating application of power into the first circuit.

20. An electronic system, comprising an integrated breathing circuit and a control device; wherein
the integrated breathing circuit comprises: a first circuit, comprising two heater wires coupled together, and a second circuit, comprising a temperature sensor and two wires connected with the temperature sensor; wherein the heater wires are configured to regulate and maintain a temperature within the integrated breathing circuit, and the temperature sensor is configured to measure the temperature within the integrated breathing circuit.
